SEQUENCE LISTING (10 sequences)

SEQ ID NO: 1 - DNA, 429 bp, Homo sapien
atggccaggtacatgctgctgctgctcctggcggtatgggtgctgaccggggagctgtggccgggagctgaggcccgggcagcgccttacggggtcaggctttgcggccgagaattcatccgagcagtcatcttcacctgcgggggctcccggtggagacgatcagacatcctggcccacgaggctatgggagataccttcccggatgcagatgctgatgaagacagtctggcaggcgagctggatgaggccatggggtccagcgagtggctggccctgaccaagtcaccccaggccttttacagggggcgacccagctggcaaggaacccctggggttcttcggggcagccgagatgtcctggctggcctttccagcagctgctgcaagtgggggtgtagcaaaagtgaaatcagtagcctttgctag

SEQ ID NO: 2 - protein, 142 aa, Homo sapien
MARYMLLLLLAVWVLTGELWPGAEARAAPYGVRLCGREFIRAVIFTCGGSRWRRSDILAHEAMGDTFPDADADEDSLAGELDEAMGSSEWLALTKSPQAFYRGRPSWQGTPGVLRGSRDVLAGLSSSCCKWGCSKSEISSLC

SEQ ID NO: 3 - DNA, 426 bp, Homo sapien
atggcaatgctcgggctgctgctgctggcttcctgggctctcctcggggctctggggctgcaggccgaggcgaggccggcgccctacggggtgaagctctgcggtcgggagttcatccgcgcggtcatcttcacttgcggaggctcacgatggcgccgggcggacatcttggcccacgaatctctgggggacttcttcgctgatggagaagccaatacagaccacctggccagcgagctggatgaagcggtgggctccagcgagtggctggccctaaccaaatccccccaggctttctacggtggtcgagccagctggcaagggtcacctggagtggttcggggcagcagagatgtgttggctggcctttccagcagttgctgcgagtggggctgtagcaagagccaaattagcagcttgtgctag

SEQ ID NO: 4 - protein, 141 aa, Mus musculus
MAMLGLLLLASWALLGALGLQAEARPAPYGVKLCGREFIRAVIFTCGGSRWRRADILAHESLGDFFADGEANTDHLASELDEAVGSSEWLALTKSPQAFYGGRASWQGSPGVVRGSRDVLAGLSSSCCEWGCSKSQISSLC

SEQ ID NO: 5 - protein, 390 aa, Artificial Sequence
  M126-Fc fusion protein
MAMLGLLLLASWALLGALGLQAEARPAPYGVKLCGREFIRAVIFTCGGSRWRRADILAHESLGDFFADGEANTDHLASELDEAVGSSEWLALTKSPQAFYGGRASWQGSPGVVRGSRDVLAGLSSSCCEWGCSKSQISSLCENENLYFQRSEPSGPTSTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCAVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 6 - protein, 239 aa, Mus musculus
EPSGPTSTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCAVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 7 - DNA, 558 bp, Homo sapien
atgcctcgcctgttcttgttccacctgctagaattctgtttactactgaaccaattttccagagcagtcgcggccaaatggaaggacgatgtcattaaattatgcggccgcgaattagttcgcgcgcagattgccatttgcggcatgagcacctggagcaaaaggtctctgagccaggaagatgctcctcagacacctagaccagtggcagaaattgtaccatccttcatcaacaaagatacagaaactataattatcatgttggaattcattgctaatttgccaccggagctgaaggcagccctatctgagaggcaaccatcattaccagagctacagcagtatgtacctgcattaaaggattccaatcttagctttgaagaatttaagaaacttattcgcaataggcaaagtgaagccgcagacagcaatccttcagaattaaaatacttaggcttggatactcattctcaaaaaaagagacgaccctacgtggcactgtttgagaaatgttgcctaattggttgtaccaaaaggtctcttgctaaatattgctga

SEQ ID NO: 8 - protein, 185 aa, Homo sapien
MPRLFLFHLLEFCLLLNQFSRAVAAKWKDDVIKLCGRELVRAQIAICGMSTWSKRSLSQEDAPQTPRPVAEIVPSFINKDTETIIIMLEFIANLPPELKAALSERQPSLPELQQYVPALKDSNLSFEEFKKLIRNRQSEAADSNPSELKYLGLDTHSQKKRRPYVALFEKCCLIGCTKRSLAKYC

SEQ ID NO: 9 - DNA, 558 bp, Homo sapien
atgcctcgcctgttttttttccacctgctaggagtctgtttactactgaaccaattttccagagcagtcgcggactcatggatggaggaagttattaaattatgcggccgcgaattagttcgcgcgcagattgccatttgcggcatgagcacctggagcaaaaggtctctgagccaggaagatgctcctcagacacctagaccagtggcagaaattgtgccatccttcatcaacaaagatacagaaaccataaatatgatgtcagaatttgttgctaatttgccacaggagctgaagttaaccctgtctgagatgcagccagcattaccacagctacaacaacatgtacctgtattaaaagattccagtcttctctttgaagaatttaagaaacttattcgcaatagacaaagtgaagccgcagacagcagtccttcagaattaaaatacttaggcttggatactcattctcgaaaaaagagacaactctacagtgcattggctaataaatgttgccatgttggttgtaccaaaagatctcttgctagattttgctga

SEQ ID NO: 10 - protein, 185 aa, Homo sapien
MPRLFFFHLLGVCLLLNQFSRAVADSWMEEVIKLCGRELVRAQIAICGMSTWSKRSLSQEDAPQTPRPVAEIVPSFINKDTETINMMSEFVANLPQELKLTLSEMQPALPQLQQHVPVLKDSSLLFEEFKKLIRNRQSEAADSSPSELKYLGLDTHSRKKRQLYSALANKCCHVGCTKRSLARFC

(19) United States
(12) Patent Application Publication
(10) Pub. No.: US 2009/0088374 A1
(43) Pub. Date: Apr. 2, 2009

(54) NOVEL USE

(76) Inventors: Judithann Lee, King of Prussia, PA (US); Qin Lu, King of Prussia, PA (US); John R. White, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 11/813,288

(22) PCT Filed: Jan. 6, 2006
(86) PCT No.: PCT/US06/00418

§ 371 Date: May 28, 2008

Related U.S. Application Data
(60) Provisional application No. 60/642,362, filed on Jan. 7, 2005.

(51) Int. Cl. A61K 38/16

(52) U.S. Cl. 514/12

(57) ABSTRACT

The present invention relates generally to the use of PA126 polypeptides and polynucleotides for healing various wounds arising from different causes.

### FIELD OF THE INVENTION

The present invention relates generally to the use of PA126 polypeptides and polynucleotides for healing various wounds arising from different causes.

### BACKGROUND OF THE INVENTION

The drug discovery process is currently undergoing a fundamental revolution as it embraces “functional genomics”, that is, high throughput genome- or gene-based biology. This approach as a means to identify genes and gene products as therapeutic targets is rapidly superceding earlier approaches based on “positional cloning”. A phenotype, that is a biological function or genetic disease, would be identified and this would then be tracked back to the responsible gene, based on its genetic map position.

Functional genomics relies heavily on high-throughput DNA sequencing technologies and the various tools of bioinformatics to identify gene sequences of potential interest from the many molecular biology databases now available. There is a continuing need to identify and characterise further genes and their related polypeptides/proteins, as targets for drug discovery.

Proteins and polypeptides that are naturally secreted into blood, lymph and other body fluids, or secreted into the cellular membrane are of primary interest for pharmaceutical research and development. The reason for this interest is the relative ease to target protein therapeutics into their place of action (body fluids or the cellular membrane). The natural pathway for protein secretion into extracellular space is the endoplasmic reticulum in eukaryotes and the inner membrane in prokaryotes (Palade, 1975, Science, 189, 347; Milstein, Brownlee, Harrison, and Mathews, 1972, Nature New Biol., 239, 117; Blobel, and Dobberstein, 1975, J. Cell. Biol., 67, 835). On the other hand, there is no known natural pathway for exporting a protein from the exterior of the cells into the cytosol (with the exception of pinocytosis, a mechanism of snake venom toxin intrusion into cells). Therefore targeting protein therapeutics into cells poses extreme difficulties.

The secreted and membrane-associated proteins include but are not limited to all peptide hormones and their receptors (including but not limited to insulin, growth hormones, chemokines, cytokines, neuropeptides, integrins, kallikreins, lamins, melanins, natriuretic hormones, neuropsin, neurotropins, pituitiary hormones, pleiotropins, prostaglandins, secretogranins, selectins, thromboglobulins, thymosins), the breast and colon cancer gene products, leptin, the obesity gene protein and its receptors, serum albumin, superoxide dismutase, spliceosome proteins, 7TM (transmembrane) proteins also called as G-protein coupled receptors, immunoglobulins, several families of serine proteinases (including but not limited to proteins of the blood coagulation cascade, digestive enzymes), deoxyribonuclease I, etc. Therapeutics based on secreted or membrane-associated proteins approved by FDA or foreign agencies include but are not limited to insulin, glucagon, growth hormone, chorionic gonadotropin, follicle stimulating hormone, luteinizing hormone, calcitonin, adrenocorticotropic hormone (ACTH), vasopressin, interleukines, interferones, immunoglobulins, lactoferrin (diverse products marketed by several companies), tissue-type plasminogen activator (Alteplase by Genentech), hyaulorindase (Wydase by Wyeth-Ayerst), dornase alpha (Pulmozyme\ by Genentech), Chymodiactin (chymopapain by Knoll), alglucerase (Ceredase by Genzyme), streptokinase (Kabikinase by Pharmacia) (Streptase by Astra), etc. This indicates that secreted and membrane-associated proteins have an established, proven history as therapeutic targets.

Our copending application WO02/22808 published 21 March, 2002 discloses a gene called sbg934114Relaxin which encodes a secreted protein. It was characterized as having homology close to mouse insulin-like peptide, Applicants have now discovered sbg934114Relaxin and its homologues have beneficial effects of treating, healing or preventing a patient with wounds caused from including, but not limited to, skin wounds, surgical wounds, burns, leg ulcers, diabetic ulcers, venous insufficiency ulcers, pressure ulcers, mucositis (both gastrointestinal and oral), renal fibrosis, lung fibrosis, COPD, or other lung diseases where damage to the epithelial cells and scar formation has occurred. Also sbg93411Relaxin and its homologues have beneficial effects of treating, healing or preventing osteoarthritis and rheumatoid arthritis; and promoting cardiovascular tissue repair following reperfusion injury.

### SUMMARY OF THE INVENTION

In one aspect, the present invention provides a method of (1) treating, healing or preventing wounds in a patient caused from including, but not limited to, skin wounds, surgical wounds, burns, leg ulcers, diabetic ulcers, venous insufficiency ulcers, pressure ulcers, mucositis (both gastrointestinal and oral), renal fibrosis, lung fibrosis, COPD, or other lung diseases where damage to the epithelial cells and scar formation has occurred; or (2) treating, healing or preventing osteoarthritis and rheumatoid arthritis; or (3) promoting cardiovascular tissue repair following reperfusion injury; the method comprising administering to the patient in need thereof an effective amount of a PA126 polypeptide or polynucleotide.

In further aspect, the invention also provides a pharmaceutical composition (formulation) for (1) treating, healing or preventing wounds in a patient caused from including, but not limited to, skin wounds, surgical wounds, burns, leg ulcers, diabetic ulcers, venous insufficiency ulcers, pressure ulcers, mucositis (both gastrointestinal and oral), renal fibrosis, lung fibrosis, COPD, or other lung diseases where damage to the epithelial cells and scar formation has occurred; or (2) treating, healing or preventing osteoarthritis and rheumatoid arthritis; or (3) promoting cardiovascular tissue repair following reperfusion injury comprising an effective amount of a PA126 polypeptide or polynucleotide and a pharmaceutically acceptable carrier.

Yet in a further aspect, the present invention relates to the use of a PA126 polypeptide or polynucleotide in the preparation of a medicament for (1) treating, healing or preventing wounds in a patient caused from including, but not limited to, skin wounds, surgical wounds, burns, leg ulcers, diabetic ulcers, venous insufficiency ulcers, pressure ulcers, mucositis (both gastrointestinal and oral), renal fibrosis, lung fibrosis, COPD, or other lung diseases where damage to the epithelial cells and scar formation has occurred; or (2) treating, healing or preventing osteoarthritis and rheumatoid arthritis; or (3) promoting cardiovascular tissue repair following reperfusion injury

### BRIEF DESCRIPTION OF THE FIGURES

FIG. 1. Topical delivery of Ad.MPA126 (SEQ ID NO:3 in adenovirus) accelerates wound closure.

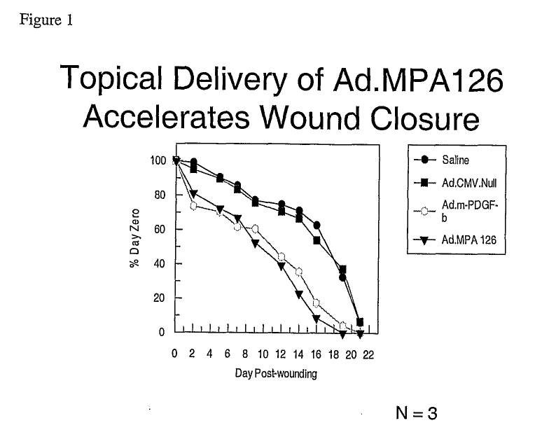

FIG. 2. Systemic delivery (i.v.) of Ad.MPA126 (SEQ ID NO:3 in adenovirus) promotes wound repair in ob/ob mice.

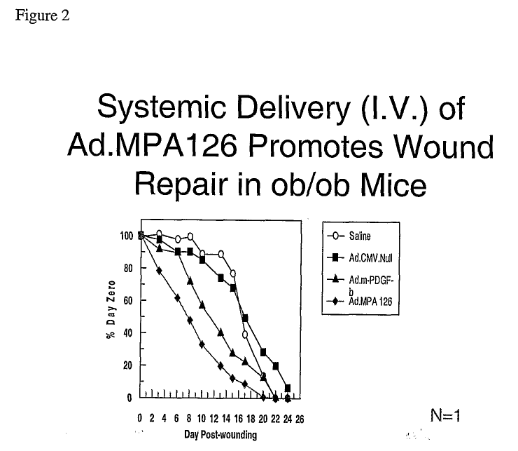

FIG. 3. Effect of topical delivery of adenovirus expressing human relaxin-1 (SEQ ID NO:7), human relaxin-2 (SEQ ID NO:9), and human relaxin-3 (SEQ ID NO:1) on wound closure in female ob/ob mice.

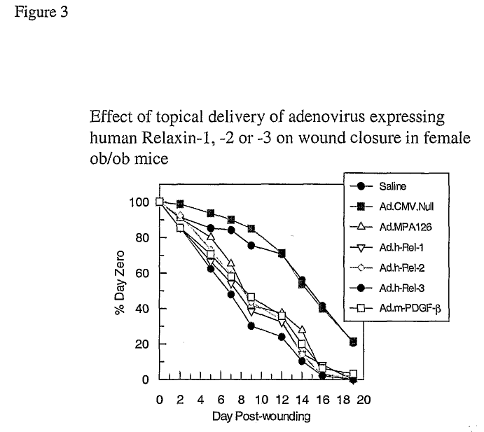

FIG. 4. MPA126-Fc fusion protein (SEQ ID NO:5) enhanced tissue repair in a dose dependent manner.

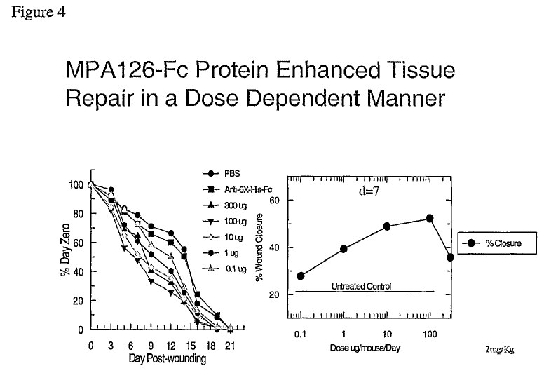

FIG. 5. HIPA126 (SEQ ID NO:1) is greatly underexpressed in diseased tissues, specifically, OA and COPD.

### DETAILED DESCRIPTION OF THE INVENTION

The following definitions are provided to facilitate understanding of certain terms and abbreviations used frequently in this application.

“Isolated” means altered “by the hand of man” from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide separated from the coexisting materials of its natural state is “isolated”, as the term is employed herein.

“Polynucleotide” generally refers to any polyribonucleotide or polydeoxyribonucleotide, which may be unmodified RNA or DNA or modified RNA or DNA. “Polynucleotides” include, without limitation, single- and double-stranded DNA, DNA that is a mixture of single- and double-stranded regions, single- and double-stranded RNA, and RNA that is mixture of single- and double-stranded regions, hybrid molecules comprising DNA and RNA that may be single-1 stranded or, more typically, double-stranded or a mixture of single- and double-stranded regions. In addition, “polynucleotide” refers to triple-stranded regions comprising RNA or DNA or both RNA and DNA. The term “polynucleotide” also includes DNAs or RNAs containing one or more modified bases and DNAs or RNAs with backbones modified for stability or for other reasons. “Modified” bases include, for example, tritylated bases and unusual bases such as inosine. A variety of modifications may be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

“Polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as post-translational processing, or by chemical modification techniques which are well known in the art. Such modifications are well described in basic texts and in more detailed monographs, as well as in a voluminous research literature. Modifications may occur anywhere in a polypeptide, including the peptide backbone, the amino acid side-chains and the amino or carboxyl termini. It will be appreciated that the same type of modification may be present to the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from post-translation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination (see, for instance, PROTEINS—STRUCTURE AND MOLECULAR PROPERTIES, 2nd Ed., T. E. Creighton, W. H. Freeman and Company, New York, 1993, Wold, F., Post-translational Protein Modifications: Perspectives and Prospects, pgs. 1-12 in POSTTRANSLATIONAL COVALENT MODIFICATION OF PROTEINS, B. C. Johnson, Ed., Academic Press, New York, 1983; Seifter et al., “Analysis for protein modifications and nonprotein cofactors”, Meth Enzymol (1990) 182:626-646 and Rattan et al., “Protein Synthesis: Post-translational Modifications and Aging”, Ann NY Acad Sci (1992) 663:48-62).

“Variant” refers to a polynucleotide or polypeptide that differs from a reference polynucleotide or polypeptide, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis.

“Identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Preferred methods to determine identity are designed to give the largest match between the sequences tested. Methods to determine identity and similarity are codified in publicly available computer programs. Preferred computer program methods to determine identity and similarity between two sequences include, but are not limited to, the GCG program package (Devereux, J., et al., Nucleic Acids Research 12(1): 387 (1984)), BLASTP, BLASTN, and FASTA (Atschul, S. F. et al., J. Molec. Biol. 215: 403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well known Smith Waterman algorithm may also be used to determine identity.

Preferred parameters for polypeptide sequence comparison include the following:

1) Algorithm: Needleman and Wunsch, J. Mol. Biol. 48: 443-453 (1970)
Comparison matrix: BLOSSUM62 from Hentikoff and Hentikoff, Proc. Natl. Acad. Sci. USA. 89:10915-10919 (1992)

### Gap Penalty: 12

### Gap Length Penalty: 4

A program useful with these parameters is publicly available as the “gap” program from Genetics Computer Group, Madison Wis. The aforementioned parameters are the default parameters for peptide comparisons (along with no penalty for end gaps).

Preferred parameters for polynucleotide comparison include the following:

1) Algorithm: Needleman and Wunsch, J. Mol. Biol. 48: 443-453 (1970)
Comparison matrix: matches=+10, mismatch=0

### Gap Penalty: 50

### Gap Length Penalty: 3

Available as: The “gap” program from Genetics Computer Group, Madison Wis. These are the default parameters for nucleic acid comparisons.

By way of example, a polynucleotide sequence of the present invention may be identical to the reference sequence of SEQ ID NO:1, that is be 100% identical, or it may include up to a certain integer number of nucleotide alterations as compared to the reference sequence. Such alterations are selected from the group consisting of at least one nucleotide deletion, substitution, including transition and transversion, or insertion, and wherein said alterations may occur at the 5′ or 3′ terminal positions of the reference nucleotide sequence or anywhere between those terminal positions, interspersed either individually among the nucleotides in the reference sequence or in one or more contiguous groups within the reference sequence. The number of nucleotide alterations is determined by multiplying the total number of nucleotides in SEQ ID NO:1 by the numerical percent of the respective percent identity (divided by 100) and subtracting that product from said total number of nucleotides in SEQ ID NO:1, or:

nₙ≦xₙ−(xₙ·y),

wherein nₙ is the number of nucleotide alterations, xₙ is the total number of nucleotides in SEQ ID NO:1, and y is, for instance, 0.70 for 70%, 0.80 for 80%, 0.85 for 85%, 0.90 for 90%, 0.95 for 95%, etc., and wherein any non-integer product of xₙ and y is rounded down to the nearest integer prior to subtracting it from xₙ. Alterations of a polynucleotide sequence encoding the polypeptide of SEQ ID NO:2 may create nonsense, missense or frameshift mutations in this coding sequence and thereby alter the polypeptide encoded by the polynucleotide following such alterations.

Similarly, a polypeptide sequence of the present invention may be identical to the reference sequence of SEQ ID NO:2, that is be 100% identical, or it may include up to a certain integer number of amino acid alterations as compared to the reference sequence such that the % identity is less than 100%. Such alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in SEQ ID NO:2 by the numerical percent of the respective percent identity (divided by 100) and then subtracting that product from said total number of amino acids in SEQ ID NO:2, or:

nₐ≦xₐ−(xₐ·y),

wherein nₐ is the number of amino acid alterations, xₐ is the total number of amino acids in SEQ ID NO:2, and y is, for instance 0.70 for 70%, 0.80 for 80%, 0.85 for 85% etc., and wherein any non-integer product of xₐ and y is rounded down to the nearest integer prior to subtracting it from xa.

“Fusion protein” refers to a protein encoded by two, often unrelated, fused genes or fragments thereof. In one example, EP-A-0 464 discloses fusion proteins comprising various portions of constant region of immunoglobulin molecules together with another human protein or part thereof. In many cases, employing an immunoglobulin Fc region as a part of a fusion protein is advantageous for use in therapy and diagnosis resulting in, for example, improved pharmacokinetic properties [see, e.g., EP-A 0232 262]. On the other hand, for some uses it would be desirable to be able to delete the Fc part after the fusion protein has been expressed, detected and purified.

### PA126 Polypeptides (Polypeptides of the Present Invention)

PA126 polypeptides include isolated polypeptides comprising an amino acid sequence which has at least 70% identity, preferably at least 80% identity, more preferably at least 90% identity, yet more preferably at least 95% identity, most preferably at least 97-99% identity, to that of SEQ ID NO:2 (HPA 126, human) or SEQ ID NO:4 (MPA126, murine) over the entire length of SEQ ID NO:2 and SEQ ID NO:4, respectively. Such polypeptides include those comprising the amino acid of SEQ ID NO:2 or SEQ ID NO:4.

Further polypeptides of the present invention include isolated polypeptides in which the amino acid sequence has at least 70% identity, preferably at least 80% identity, more preferably at least 90% identity, yet more preferably at least 95% identity, most preferably at least 97-99% identity, to the amino acid sequence of SEQ ID NO:2 or 4 over the entire length of SEQ ID NO:2 or 4, respectively. Such polypeptides include the polypeptide of SEQ ID NO:2 or 4.

Further polypeptides of the present invention include isolated polypeptides encoded by a polynucleotide comprising the sequence contained in SEQ ID NO:1 or 3.

Polypeptides of the present invention are now found to be active in (1) treating, healing or preventing wounds in a patient caused from including, but not limited to, skin wounds, surgical wounds, burns, leg ulcers, diabetic ulcers, venous insufficiency ulcers, pressure ulcers, mucositis (both gastrointestinal and oral), renal fibrosis, lung fibrosis, COPD, or other lung diseases where damage to the epithelial cells and scar formation has occurred; or (2) treating, healing or preventing osteoarthritis and rheumatoid arthritis; or (3) promoting cardiovascular tissue repair following reperfusion injury. This property is hereinafter referred to as “PA126 activity” or “PA126 polypeptide activity” or “biological activity of PA126”. Also included amongst “PA126 activity” or “PA126 polypeptide activity” or “biological activity of PA126” are antigenic and immunogenic activities of said PA126 polypeptides, in particular the antigenic and immunogenic activities of the polypeptides of SEQ ID NO:2 and SEQ ID NO:4. Preferably, a polypeptide of the present invention exhibits at least one biological activity of PA126.

The polypeptides of the present invention may be in the form of the “mature” protein or may also form a fusion protein. It is often advantageous to include an additional amino acid sequence which contains secretary or leader sequences, pro-sequences, sequences which aid in purification such as multiple histidine residues, or an additional sequence for stability during recombinant production. The polypeptides of the present invention may be formed from the aforementioned polypeptide conjugated to Fc portion of an antibody, as exemplified in Example 2. Such Fc fusion proteins also have the activity to (1) treat, heal or prevent wounds in a patient caused from including, but not limited to, skin wounds, surgical wounds, burns, leg ulcers, diabetic ulcers, venous insufficiency ulcers, pressure ulcers, mucositis (both gastrointestinal and oral), renal fibrosis, lung fibrosis, COPD, or other lung diseases where damage to the epithelial cells and scar formation has occurred, or (2) treat, heal or prevent osteoarthritis and rheumatoid arthritis; or (3) promote cardiovascular tissue repair following reperfusion injury. Construction of Fc fusion proteins are well known, see for example: Aruffo, A. (1999) Immunoglobulin fusion proteins. In Antibody Fusion Proteins (S. M. Chamow, and A. Ashkenazi, Eds), Chapter 8, pp 221-241, Wiley-Liss, Inc.; Avi Ashkenazi and Steven M Chamow, Current Opinion in Immunology, 1997, 9:195-200.

Apart from Fc fusion proteins, the polypeptides of the present invention may be formed by conjugating the aforementioned polypeptides with albumin or albumin binding peptide or can even be pegylated. Conjugation with albumin or albumin binding peptide or pegylation techniques are well known, see for example: J. M. Harris and R. B. Chess, Nature Review Drug Discovery, Vol 2, pp 214-221; R. B. Greenwald et al., Advanced Drug Delivery Reviews 55 (2003) 217-250; M. S. Dennis et al., The Journal of Biological Chemistry, Vol 277, No. 38, 2002, pp 35035-35043; S. Syed et al., Blood, Vol 89, No 9, 1997: pp 3243-3252.

The polypeptides of present invention also includes variants of the aforementioned polypeptides, that is polypeptides that vary from the referents by conservative amino acid substitutions, whereby a residue is substituted by another with like characteristics. Typical such substitutions are among Ala, Val, Leu and Ile; among Ser and Thr; among the acidic residues Asp and Glu; among Asn and Gln; and among the basic residues Lys and Arg; or aromatic residues Phe and Tyr. Particularly preferred are variants in which several, 5-10, 1-5, 1-3, 1-2 or 1 amino acids are substituted, deleted, or added in any combination.

Polypeptides of the present invention can be prepared in any suitable manner. Such polypeptides include isolated naturally occurring polypeptides, recombinantly produced polypeptides, synthetically produced polypeptides, or polypeptides produced by a combination of these methods. Means for preparing such polypeptides are well understood in the art.

Recombinant polypeptides of the present invention may be prepared by processes well known in the art from genetically engineered host cells comprising expression systems. Accordingly, in a further aspect, the present invention relates to expression systems which comprises a polynucleotide or polynucleotides encoding the polypeptides of the present invention, to host cells which are genetically engineered with such expression systems and to the production of polypeptides of the invention by recombinant techniques. Cell-free translation systems can also be employed to produce such proteins using RNAs derived from the DNA constructs of the present invention.

Representative examples of appropriate hosts include bacterial cells, such as streptococci, staphylococci, E. coli, Streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, HEK 293 and Bowes melanoma cells; and plant cells.

A great variety of expression systems can be used, for instance, chromosomal, episomal and virus-derived systems, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression systems may contain control regions that regulate as well as engender expression. Generally, any system or vector which is able to maintain, propagate or express a polynucleotide to produce a polypeptide in a host may be used. The appropriate nucleotide sequence may be inserted into an expression system by any of a variety of well-known and routine techniques, such as, for example, those set forth in Sambrook et al., Molecular Cloning: A Laboratory Manual, 2nd Ed., Cold Spring Harbor Laboratory Press, Cold Spring Harbor, N.Y. (1989). Appropriate secretion signals may be incorporated into the desired polypeptide to allow secretion of the translated protein into the lumen of the endoplasmic reticulum, the periplasmic space or the extracellular environment. These signals may be endogenous to the polypeptide or they may be heterologous signals.

Polypeptides of the present invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, high performance liquid chromatography, hydroxylapatite chromatography and lectin chromatography. Most preferably, affinity chromatography is employed for purification. Well-known techniques for refolding proteins may be employed to regenerate active conformation when the polypeptide is denatured during isolation and or purification.

The polypeptides of the present invention can be formulated into pharmaceutical compositions and administered in the same manner as described for other polypeptides. See, e.g., International Patent Application, Publication No. WO90/02762. Generally, these compositions contain a therapeutically effective amount of a polypeptide of this invention and an acceptable pharmaceutical carrier. Suitable carriers are well known to those of skill in the art and include, for example, saline. Alternatively, such compositions may include conventional delivery systems into which polypeptide of the invention is incorporated. Optionally, these compositions may contain other active ingredients.

### PA126 Polynucleotides (Polynucleotides of the Present Invention)

In one aspect, the present invention relates to PA-126 polynucleotides. Such polynucleotides include isolated polynucleotides comprising a nucleotide sequence encoding a polypeptide which has at least 70% identity, preferably at least 80% identity, more preferably at least 90% identity, yet more preferably at least 95% identity, to the amino acid sequence of SEQ ID NO:2 or 4, over the entire length of SEQ ID NO:2 or 4, respectively. In this regard, polypeptides which have at least 97% identity are highly preferred, whilst those with at least 98-99% identity are more highly preferred, and those with at least 99% identity are most highly preferred. Such polynucleotides include a polynucleotide comprising the nucleotide sequence contained in SEQ ID NO:1 encoding the polypeptide of SEQ ID NO:2, or a polynucleotide comprising the nucleotide sequence contained in SEQ ID NO:3 encoding the polypeptide of SEQ ID NO:4.

Further polynucleotides of the present invention include isolated polynucleotides comprising a nucleotide sequence which has at least 70% identity, preferably at least 80% identity, more preferably at least 90% identity, yet more preferably at least 95% identity, to SEQ ID NO:1 or 3 over the entire length of SEQ ID NO:1 or 3, respectively. In this regard, polynucleotides which have at least 97% identity are highly preferred, whilst those with at least 98-99% identify are more highly preferred, and those with at least 99% identity are most highly preferred. Such polynucleotides include a polynucleotide comprising the polynucleotide of SEQ ID NO: 1 or 3 as well as the polynucleotide of SEQ ID NO:1 or 3.

The polynucleotides of the present invention also include any other polynucleotides which encodes the polypeptides of the present invention.

The invention also provides polynucleotides which are complementary to all the above described polynucleotides.

The polypeptides of this invention may be administered by any appropriate internal route, and may be repeated as needed, e.g., as frequently as one to three times daily for between 1 day to about three weeks to once per week or once biweekly. The dose and duration of treatment relates to the relative duration of the molecules of the present invention in the human circulation, and can be adjusted by one of skilled in the art depending upon the condition being treated and the general health of the patient.

As used herein, the term “pharmaceutical” includes veterinary applications of the invention. The term “therapeutically effective amount” refers to that amount of therapeutic agent, which is useful for alleviating a selected condition.

In a specific embodiment, polynucleotides (nucleic acid sequences or simply as nucleic acids) comprising polynucleotide sequences encoding the instant polypeptides of the invention or functional derivatives thereof are administered to treat a wound by way of gene therapy. “Gene therapy” refers to therapy performed by the administration to a subject of an expressed or expressible nucleic acid. In this embodiment of the invention, the nucleic acids produce their encoded protein that mediates a therapeutic effect.

Any of the methods for gene therapy available in the art can be used according to the present invention. Exemplary methods are described below.

For general reviews of the methods of gene therapy, see Goldspiel et al., Clinical Pharmacy 12:488-505 (1993); Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, TIBTECH 11(5):155-215 (1993). Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a preferred aspect, the nucleic acid sequences encoding a polypeptide, said nucleic acid sequences form a part of expression vectors that express a polypeptide in a suitable host. In particular, such nucleic acid sequences have promoters operably linked to the polypeptide coding region, said promoter being inducible or constitutive, and, optionally, tissue-specific. In another particular embodiment, nucleic acid molecules are used in which the polypeptide coding sequences and any other desired sequences are flanked by regions that promote homologous recombination at a desired site in the genome, thus providing for intrachromosomal expression of the a polypeptide-encoding nucleic acids (Koller and Smithies, Proc. Natl. Acad. Sci. USA 86:8932-8935 (1989); Zijlstra et al., Nature 342:435-438 (1989).

Delivery of the nucleic acids into a patient may be either direct, in which case the patient is directly exposed to the nucleic acid or nucleic acid-carrying vectors, or indirect, in which case, cells are first transformed with the nucleic acids in vitro, then transplanted into the patient. These two approaches are known, respectively, as in vivo or ex vivo gene therapy.

In a specific embodiment, the nucleic acid sequences are directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, J. Biol. Chem. 262:4429-4432 (1987)) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180; WO 92/22635; WO92/20316; WO93/14188, WO 93/20221). Alternatively, the nucleic acid can be introduced intracellularly and incorporated within host cell DNA for expression, by homologous recombination (Koller and Smithies, Proc. Natl. Acad. Sci. USA 86:8932-8935 (1989); Zijlstra et al., Nature 342:435-438 (1989)).

In a specific embodiment, viral vectors that contain nucleic acid sequences encoding a polypeptide of the invention are used. For example, a retroviral vector can be used (see Miller et al., Meth. Enzymol. 217:581-599 (1993)). These retroviral vectors contain the components necessary for the correct packaging of the viral genome and integration into the host cell DNA. The nucleic acid sequences encoding the polypeptides of the present invention to be used in gene therapy are cloned into one or more vectors, which facilitates delivery of the gene into a patient. More detail about retroviral vectors can be found in Boesen et al., Biotherapy 6:291-302 (1994), which describes the use of a retroviral vector to deliver the mdr1 gene to hematopoietic stem cells in order to make the stem cells more resistant to chemotherapy. Other references illustrating the use of retroviral vectors in gene therapy are: Clowes et al., J. Clin. Invest. 93:644-651 (1994); Kiem et al., Blood 83:1467-1473 (1994); Salmons and Gunzberg, Human Gene Therapy 4:129-141 (1993); and Grossman and Wilson, Curr. Opin. in Genetics and Devel. 3:110-114 (1993).

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, Current Opinion in Genetics and Development 3:499-503 (1993) present a review of adenovirus-based gene therapy. Bout et al., Human Gene Therapy 5:3-10 (1994) demonstrated the use of adenovirus vectors to transfer genes to the respiratory epithelia of rhesus monkeys. Other instances of the use of adenoviruses in gene therapy can be found in Rosenfeld et al., Science 252:431-434 (1991); Rosenfeld et al., Cell 68:143-155 (1992); Mastrangeli et al., J. Clin. Invest. 91:225-234 (1993); PCT Publication WO94/12649; and Wang, et al., Gene Therapy 2:775-783 (1995). In a preferred embodiment, adenovirus vectors are used. Adeno-associated virus (AAV) has also been proposed for use in gene therapy (Walsh et al., Proc. Soc. Exp. Biol. Med. 204:289-300 (1993); U.S. Pat. No. 5,436,146).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, Meth. Enzymol. 217:599-618 (1993); Cohen et al., Meth. Enzymol. 217:618-644 (1993); Cline, Pharmac. Ther. 29:69-92m (1985) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T-lymphocytes, B-lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a preferred embodiment, the cell used for gene therapy is autologous to the patient.

In an embodiment in which recombinant cells are used in gene therapy, nucleic acid sequences encoding a polypeptide are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention, (see e.g. PCT Publication WO 94/08598; Stemple and Anderson, Cell 7.1:973-985 (1992); Rheinwald, Meth. Cell Bio. 21A:229 (1980); and Pittelkow and Scott, Mayo Clinic Proc. 61:771 (1986)).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

The invention provides methods of treatment, inhibition and prophylaxis by administration to a subject of an effective amount of a polypeptide or a polynucleotide of the present invention (hereinafter sometimes referred to as a “compound”) or pharmaceutical composition of the present invention. In a preferred aspect, the compound is substantially purified (e.g., substantially free from substances that limit its effect or produce undesired side-effects). The subject is preferably an animal, including but not limited to animals such as cows, pigs, horses, chickens, cats, dogs, etc., and is preferably a mammal, and most preferably human.

Formulations and methods of administration that can be employed when the compound comprises a polynucleotide or a polypeptide are described above; additional appropriate formulations and routes of administration can be selected from among those described herein below.

Various delivery systems are known and can be used to administer a compound of the invention, e.g., encapsulation in liposomes, microparticles, microcapsules, recombinant cells capable of expressing the compound, receptor-mediated endocytosis (see, e.g., Wu and Wu, J. Biol. Chem. 262:4429-4432 (1987)), construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compounds or compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, of the invention, care must be taken to use materials to which the protein does not absorb.

In another embodiment, the compound or composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the compound or composition can be delivered in a controlled release system. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)).

Other controlled release systems are discussed in the review by Langer (Science 249:1527-1533 (1990)).

In a specific embodiment where the compound of the invention is a nucleic acid encoding a polypeptide, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., Proc. Natl. Acad. Sci. USA 88:1864-1868 (1991)), etc. Alternatively, a nucleic acid can be introduced intracellularly and incorporated within host cell DNA for expression, by homologous recombination.

The present invention also provides, pharmaceutical compositions (formulations). Such compositions comprise, a therapeutically effective amount of a compound, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Rernington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The amount of the compound of the invention which will be effective in the treatment, inhibition and prevention of a disease or disorder associated with aberrant expression and/or activity of a polypeptide of the invention can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems.

For polypeptides, the dosage administered to a patient is typically 0.1 mg/kg to 100 mg/kg of the patient's body weight. Preferably, the dosage administered to a patient is between 0.1 mg/kg and 20 mg/kg of the patient's body weight, more preferably 1 mg/kg to 10 mg/kg of the patient's body weight. Generally, human polypeptides have a longer half-life within the human body than polypeptides from other species due to the immune response to the foreign polypeptides. Thus, lower dosages of human polypeptides and less frequent administration is often possible. Further, the dosage and frequency of administration of polypeptides of the invention may be reduced by enhancing uptake and tissue penetration (e.g., into the brain) of the polypeptides by modifications such as, for example, lipidation.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

This invention provides for a pharmaceutical composition which comprises a polypeptide of this invention and a pharmaceutically acceptable carrier, diluent or excipient. Accordingly, the polypeptide may be used in the manufacture of a medicament. Pharmaceutical compositions of the invention may be formulated as solutions or lyophilized powders for parenteral administration. Powders may be reconstituted by addition of a suitable diluent or other pharmaceutically acceptable carrier prior to use. The liquid formulation may be a buffered, isotonic, aqueous solution. Examples of suitable diluents are normal isotonic saline solution, standard 5% dextrose in water or buffered sodium or ammonium acetate solution. Such formulation is especially suitable for parenteral administration, but may also be used for oral administration or contained in a metered dose inhaler or nebulizer for insufflation. It may be desirable to add excipients such as polyvinylpyrrolidone, gelatin, hydroxy cellulose, acacia, polyethylene glycol, mannitol, sodium chloride or sodium citrate.

Alternately, the polypeptide may be encapsulated, tableted or prepared in an emulsion or syrup for oral administration. Pharmaceutically acceptable solid or liquid carriers may be added to enhance or stabilize the composition, or to facilitate preparation of the composition. Solid carriers include starch, lactose, calcium sulfate dihydrate, terra alba, magnesium stearate or stearic acid, talc, pectin, acacia, agar or gelatin. Liquid carriers include syrup, peanut oil, olive oil, saline and water. The carrier may also include a sustained release material such as glyceryl monostearate or glyceryl distearate, alone or with a wax. The amount of solid carrier varies but, preferably, will be between about 20 mg to about 1 g per dosage unit. The pharmaceutical preparations are made following the conventional techniques of pharmacy involving milling, mixing, granulating, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The mode of administration of a polypeptide of the invention may be any suitable route which delivers the agent to the host. The polypeptides and pharmaceutical compositions of the invention are particularly useful for parenteral administration, i.e., subcutaneously, intramuscularly, intravenously or intranasally.

Polypeptide of the invention may be prepared as pharmaceutical compositions containing an effective amount of a polypeptide of the invention as an active ingredient in a pharmaceutically acceptable carrier. In the compositions of the invention, an aqueous suspension or solution containing the polypeptide, preferably buffered at physiological pH, in a form ready for injection is preferred. The compositions for parenteral administration will commonly comprise a solution of the polypeptide of the invention or a cocktail thereof dissolved in an pharmaceutically acceptable carrier, preferably an aqueous carrier. A variety of aqueous carriers may be employed, e.g., 0.4% saline, 0.3% glycine and the like. These solutions are sterile and generally free of particulate matter. These solutions may be sterilized by conventional, well known sterilization techniques (e.g., filtration). The compositions may contain pharmaceutically acceptable auxiliary substances as required to approximate physiological conditions such as pH adjusting and buffering agents, etc. The concentration of the polypeptide of the invention in such pharmaceutical formulation can vary widely, i.e., from less than about 0.5%, usually at or at least about 1% to as much as 15 or 20% by weight and will be selected primarily based on fluid volumes, viscosities, etc., according to the particular mode of administration selected.

Thus, a pharmaceutical composition of the invention for intramuscular injection could be prepared to contain 1 mL sterile buffered water, and between about 1 ng to about 100 mg, e.g. about 50 ng to about 30 mg or more preferably, about 5 mg to about 25 mg, of a polypeptide of the invention. Similarly, a pharmaceutical composition of the invention for intravenous infusion could be made up to contain about 250 mL of sterile Ringer's solution, and about 1 mg to about 30 mg and preferably 5 mg to about 25 mg of a polypeptide of the invention. Actual methods for preparing parenterally administrable compositions are well known or will be apparent to those skilled in the art and are described in more detail in, for example, “Remington's Pharmaceutical Science”, 15th ed., Mack Publishing Company, Easton, Pa.

It is preferred that the polypeptide of the invention, when in a pharmaceutical preparation, be present in unit dose forms. The appropriate therapeutically effective dose can be determined readily by those of skill in the art. Such dose may, if necessary, be repeated at appropriate time intervals selected as appropriate by a physician during the response period.

The present invention may be embodied in other specific forms, without departing from the spirit or essential attributes thereof, and, accordingly, reference should be made to the appended claims, rather than to the foregoing specification or following examples, as indicating the scope of the invention.

### EXAMPLES

### Example 1

### Generation of Adenovirus

Adenovirus MPA-126 (relaxin) was generated as follows. The ORF for MPA-126 (SEQ ID NO:3) was subcloned into the adenovirus shuttle vector pShuttle (ClonTech) using appropriate restriction sites, placing the ORF downstream of the CMV IE promoter in the correct orientation. An I-CeuI/PI-SceI fragment containing the expression cassette (CMV IE-ORF-BGH polyA) was isolated from the shuttle vector and was swapped with a GFP expression cassette driven by bacterial Lac promoter at the I-CeuI/PI-SceI sites of the adenovirus backbone plasmid pAdX. The cloning step was carried by a convenient green/white selection process, in which white colonies contained the recombinant construct, pAdX.MPA-126. The purified molecular clone DNA of j adenovirus vector was linearized by digesting with restriction enzyme PacI to expose ITRs, and transfected into HEK293 cells for adenovirus rescue. The adenovirus was amplified and purified by CsCl banding as described (Engelhardt, J. 1999. Methods for adenovirus-mediated gene transfer to airway epithelium. In Methods in Molecular Medicine, Gene Therapy Protocols, P. Robbins (Ed.). p. 169-184. Humana Press, Totowa). Concentrated adenovirus was desalted by using a sterilized Bio-gel column (Bio-Rad) and stored in 1×PBS with 10% glycerol at −80° C.

The control adenovirus Ad.mPDGF-b was generated by using a direct cloning approach (Sukmanm A. J., Kallarakal, A., Fornwald, J., Kozarsky, K. F., Appelbaum, E., Shatzman, A. R., and Lu, Q. 2002. Generation of recombinant adenovirus vectors by a direct cloning approach. In Gene Cloning and Expression Technologies, M. P. Weiner and Q. Lu (Eds.). p 341-355. Eaton Publishing, Westborough, Mass.). Briefly, the ORF for murine PDGF-B was PCR amplified and cloned into the XbaI/SwaI sites of pAC2XS, placing the gene under the control of CMV IE promoter. The purified molecular clone DNA of adenovirus vector was linearized by digesting with restriction enzyme PacI to expose ITRs, and tranfected into HEK293 cells for adenovirus rescue. The adenovirus was amplified and purified by CsCl banding as described (Engelhardt, J. 1999. Methods for adenovirus-mediated gene transfer to airway epithelium. In Methods in Molecular Medicine, Gene Therapy Protocols, P. Robbins (Ed.). p. 169-184. Humana Press, Totowa). Concentrated adenovirus was desalted by using a Bio-gel column (Bio-Rad) and stored in 1×PBS with 10% glycerol at −80° C.

### Example 2

### Generation of MPA126-Fc Fusion Protein (SEQ ID NO:5)

Note that between MPA126 and Fc there is a TEV cleavage site (ENLYFQ) engineered. In the fusion, mIgg2bfc (mouse) sequence (SEQ ID NO:6) is contained between aa152E to the end aa390K. The expression construct for CHO E1A expression is constructed as follows. The ORF for the full length MPA126 (except the stop codon) was PCR amplified with codons for the tev protease cleavage site added at the C-terminus. The PCR fragment was inserted into the EcoRI/BglII sites of pIgg2bfclink to generate the MPA126-tev-mFc fusion (SEQ ID NO:5).

### Example 3

### Excisonal Wound Repair Model

Diabetic mice, such as the ob/ob strain, display delayed wound healing.¹ Ob/ob mice are a naturally occurring strain of mice that have a deletion of the ob/ob gene, which codes for leptin. Leptin binds to a cytokine class I receptor, obRb, and activates the intracellular signalling cascade which curtails appetite. Since the ob/ob mice cannot produce leptin, they are obese, being twice the weight of a normal C57/B16 mouse. The obese mice also have other metabolic defects, including reduced thermogenesis, hyperphagia, decreased fertility, and inhibition of growth hormone.² The pronounced retardation on wound healing in ob/ob mice has been attributed to their diabetic-like phenotype. Models of impaired wound healing permit the opportunity to explore the effect of specific cytokines and growth factors on wound wound repair. Topical application of the growth factor PDGG has been shown to enhance wound healing in the diabetic mouse strain, db/db.³ The db/db strain is phenotypically similar to the ob/ob strain, but the db/db mice lack the leptin receptor. The wounds of db/db mice exhibit a marked delay in cellular infiltration, granulation tissue formation, and delayed wound healing. Platelet-derived growth factor (PDGF) is both a mitogen and a chemoattractant for smooth muscle cells and fibroblasts, and caused rapid re-epithelialization of wounds in db/db mice. The novel protein, MPA126 has been demonstrated to enhanced wound closure by both topical and systemic delivery in the ob/ob wound repair model.

### Topical Delivery Experimental Design:

To determine the effect of topical delivery of MPA126 or a positive control protein, ie. PDGF, on wound repair, ten to fourteen week old female ob/ob mice were anesthesitized using a Ketamine (90 mg/kg)/Xylazine (10 mg/kg) cocktail. The upper back of the mouse was shaved, and a sterile field was established using alternate wipes of alcohol and Betadine. Full-thickness circular excisional wounds 6 mm in diameter were created using a sterile biopsy punch, resulting in two wounds per mouse. For topical delivery, adenovirus (1×10¹⁰ viral particles/wound) coding for MPA126, murine PDGF or a control empty adenovirus were applied directly onto the wounded area. A saline control was also directly applied to the wounds. Polaxamer (Pluronic F127 in 10% PBS) was subsequently overlaid onto the wounds, which were then covered with a transparent sterile dressing. To determine the rate of wound closure, the circumference of the wounds were traced onto transparency film at two day intervals. At the end of the study when all the wounds had healed, the transparency films were optically scanned, and the surface area was determined using Scion Image software (Scion Corporation, Frederick, Md., U.S.A.).

### Systemic Delivery Experimental Design:

To determine the effect of systemic delivery of MPA126, ten to fourteen week old female ob/ob mice were anesthesitized using a Ketamine (90 mg/kg)/Xylazine (10 mg/kg) cocktail. Two hours prior to the wounding procedure, the mice were given intraperitoneal injections of the MPA126-Fc protein at multiple concentrations (0.1 ug/0.5 ml to 100 ug/0.5 ml) or the vehicle (PBS without calcium and magnesium). The upper back of the mouse was shaved, and a sterile field was established using alternate wipes of alcohol and Betadine. Full-thickness circular excisional wounds 6 mm in diameter were created using a sterile biopsy punch, resulting in two wounds per mouse. Saline was applied directly onto the wounds, which were then covered with a transparent sterile dressing. To determine the rate of wound closure, the circumference of the wounds were traced onto transparency film at two day intervals. At the end of the study when all the wounds had healed, the transparency films were optically scanned, and the surface area was determined using Scion Image software (Scion Corporation, Frederick, Md., U.S.A.). Throughout the duration of the systemic studies, mice were monitored for weight loss or gain.
1.) Stallmeyer, B. et al. (2001). Systemically and topically supplemented leptin fails to reconstitute a normal angiogenic response during skin repair in diabetic ob/ob mice. Diabetologia 44: 471-479.
2.) Ring, B. D. et al. (2000). Systemically and Topically Administered Leptin Both Accelerate Wound Healing in Diabetic ob/ob Mice. Endocrinol. 141(1): 446-449
3.) Greenlaugh, D. G. et al. (1990). PDGF and FGF stimulate wound healing in the generically diabetic mouse. Am. J. Pathol. 136: 1235-1246.

### Example 4

### Expression Studies

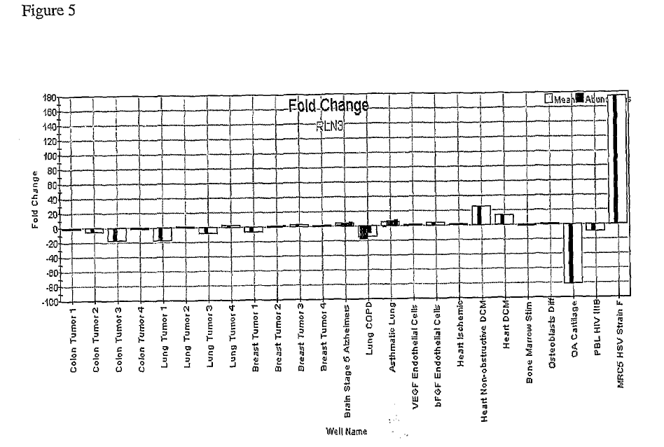

The expression data which showed that HPA126 (SEQ ID NO: 1) was underexpressed in Osteoarthritis (OA) and COPD (see FIG. 5). Both diseases involve faulty tissue repair mechanisms, resulting in “wounded” tissue. It is postulated that such underexpression of HPA126 may be a causative factor in those diseases. Thus in one aspect the present invention relates to the prevention or treatment of OA and COPD using the polynucleotides and polypeptides of the present invention.

SEQ ID NO:1:
ATGGCCAGGTACATGCTGCTGCTGCTCCTGGCGGTATGGGTGCTGACCGG
GGAGCTGTGGCCGGGAGCTGAGGCCCGGGCAGCGCCTTACGGGGTCAGGC
TTTGCGGCCGAGAATTCATCCGAGCAGTCATCTTCACCTGCGGGGGCTCC
CGGTGGAGACGATCAGACATCCTGGCCCACGAGGCTATGGGAGATACCTT
CCCGGATGCAGATGCTGATGAAGACAGTCTGGCAGGCGAGCTGGATGAGG
CCATGGGGTCCAGCGAGTGGCTGGCCCTGACCAAGTCACCCCAGGCCTTT
TACAGGGGGCGACCCAGCTGGCAAGGAACCCCTGGGGTTCTTCGGGGCAG
CCGAGATGTCCTGGCTGGCCTTTCCAGCAGCTGCTGCAAGTGGGGGTGTA
GCAAAAGTGAAATCAGTAGCCTTTGCTAG
SEQ ID NO:2:
MARYMLLLLL AVWVLTGELW PGAEARAAPY GVRLCGREFI
RAVIFTCGGS RWRRSDILAH EAMGDTFPDA DADEDSLAGE
LDEAMGSSEW LALTKSPQAF YRGRPSWQGT PGVLRGSRDV
LAGLSSSCCK WGCSKSEISS LC
SEQ ID NO:3
ATGGCAATGCTCGGGCTGCTGCTGCTGGCTTCCTGGGCTCTCCTCGGGGC
TCTGGGGCTGCAGGCCGAGGCGAGGCCGGCGCCCTACGGGGTGAAGCTCT
GCGGTCGGGAGTTCATCCGCGCGGTCATCTTCACTTGCGGAGGCTCACGA
TGGCGCCGGGCGGACATCTTGGCCCACGAATCTCTGGGGGACTTCTTCGC
TGATGGAGAAGCCAATACAGACCACCTGGCCAGCGAGCTGGATGAAGCGG
TGGGCTCCAGCGAGTGGCTGGCCCTAACCAAATCCCCCCAGGCTTTCTAC
GGTGGTCGAGCCAGCTGGCAAGGGTCACCTGGAGTGGTTCGGGGCAGCAG
AGATGTGTTGGCTGGCCTTTCCAGCAGTTGCTGCGAGTGGGGCTGTAGCA
AGAGCCAAATTAGCAGCTTGTGCTAG
SEQ ID NO:4
MAMLGLLLLA SWALLGALGL QAEARPAPYG VKLCGREFIR
AVIFTCGGSR WRRADILAHE SLGDFFADGE ANTDHLASEL
DEAVGSSEWL ALTKSPQAFY GGRASWQGSP GVVRGSRDVL
AGLSSSCCEW GCSKSQISSL C
M126-Fc Fusion Protein
(SEQ ID NO:5)
MAMLGLLLLASWALLGALGLQAEARPAPYGVKLCGREFIRAVIFTCGGSR
WRRADILAHESLGDFFADGEANTDHLASELDEAVGSSEWLALTKSPQAFY
GGRASWQGSPGVVRGSRDVLAGLSSSCCEWGCSKSQISSLCENENLYFQR
SEPSGPTSTINPCPPCKECHKCPAPNLEGGPSVFIFPPNIKDVLMISLTP
KVTCVVVDVSEDDPDVRISWFVNNVEVHTAQTQTHREDYNSTIRVVSALP
IQHQDWMSGKEFKCAVNNKDLPSPIERTISKIKGLVRAPQVYILPPPAEQ
LSRKDVSLTCLVVGFNPGDISVEWTSNGHTEENYKDTAPVLDSDGSYFIY
SKLDIKTSKWEKTDSFSCNVRHEGLKNYYLKKTISRSPGK.
mIgg2bfc protein sequence
(SEQ ID NO:6)
EPSGPTSTINPCPPCKECHKCPAPNLEGGPSVFIFPPNIKDVLMISLTPK
VTCVVVDVSEDDPDVRISWFVNNVEVHTAQTQTHREDYNSTIRVVSALPI
QHQDWMSGKEFKCAVNNKDLPSPIERTISKIKGLVRAPQVYILPPPAEQL
SRKDVSLTCLVVGFNPGDISVEWTSNGHTEENYKDTAPVLDSDGSYFIYS
KLDIKTSKWEKTDSFSCNVRHEGLKNYYLKKTISRSPGK.
Human Relaxin-1 DNA
(SEQ ID NO:7)
ATGCCTCGCCTGTTCTTGTTCCACCTGCTAGAATTCTGTTTACTACTGAA
CCAATTTTCCAGAGCAGTCGCGGCCAAATGGAAGGACGATGTCATTAAAT
TATGCGGCCGCGAATTAGTTCGCGCGCAGATTGCCATTTGCGGCATGAGC
ACCTGGAGCAAAAGGTCTCTGAGCCAGGAAGATGCTCCTCAGACACCTAG
ACCAGTGGCAGAAATTGTACCATCCTTCATCAACAAAGATACAGAAACTA
TAATTATCATGTTGGAATTCATTGCTAATTTGCCACCGGAGCTGAAGGCA
GCCCTATCTGAGAGGCAACCATCATTACCAGAGCTACAGCAGTATGTACC
TGCATTAAAGGATTCCAATCTTAGCTTTGAAGAATTTAAGAAACTTATTC
GCAATAGGCAAAGTGAAGCCGCAGACAGCAATCCTTCAGAATTAAAATAC
TTAGGCTTGGATACTCATTCTCAAAAAAAGAGACGACCCTACGTGGCACT
GTTTGAGAAATGTTGCCTAATTGGTTGTACCAAAAGGTCTCTTGCTAAAT
ATTGCTGA
Human Relaxin 1 Protein
(SEQ ID NO:8)
MPRLFLFHLLEFCLLLNQFSRAVAAKWKDDVIKLCGRELVRAQIAICGMS
TWSKRSLSQEDAPQTPRPVAEIVPSFINKDTETIIIMLEFIANLPPELKA
ALSERQPSLPELQQYVPALKDSNLSFEEFKKLIRNRQSEAADSNPSELKY
LGLDTHSQKKRRPYVALFEKCCLIGCTKRSLAKYC.
Human Relaxin 2 DNA
(SEQ ID NO:9)
ATGCCTCGCCTGTTTTTTTTCCACCTGCTAGGAGTCTGTTTACTACTGAA
CCAATTTTCCAGAGCAGTCGCGGACTCATGGATGGAGGAAGTTATTAAAT
TATGCGGCCGCGAATTAGTTCGCGCGCAGATTGCCATTTGCGGCATGAGC
ACCTGGAGCAAAAGGTCTCTGAGCCAGGAAGATGCTCCTCAGACACCTAG
ACCAGTGGCAGAAATTGTGCCATCCTTCATCAACAAAGATACAGAAACCA
TAAATATGATGTCAGAATTTGTTGCTAATTTGCCACAGGAGCTGAAGTTA
ACCCTGTCTGAGATGCAGCCAGCATTACCACAGCTACAACAACATGTACC
TGTATTAAAAGATTCCAGTCTTCTCTTTGAAGAATTTAAGAAACTTATTC
GCAATAGACAAAGTGAAGCCGCAGACAGCAGTCCTTCAGAATTAAAATAC
TTAGGCTTGGATACTCATTCTCGAAAAAAGAGACAACTCTACAGTGCATT
GGCTAATAAATGTTGCCATGTTGGTTGTACCAAAAGATCTCTTGCTAGAT
TTTGCTGA
Human Relaxin 2 Protein
(SEQ ID NO:10)
MPRLFFFHLLGVCLLLNQFSRAVADSWMEEVIKLCGRELVRAQIAICGMS
TWSKRSLSQEDAPQTPRPVAEIVPSFINKDTETINMMSEFVANLPQELKL
TLSEMQPALPQLQQHVPVLKDSSLLFEEFKKLIRNRQSEAADSSPSELKY
LGLDTHSRKKRQLYSALANKCCHVGCTKRSLARFC.

What is claimed is:

1. A method of (1) treating, healing or preventing wounds, osteoarthritis or rheumatoid arthritis; or (2) promoting cardiovascular tissue repair following reperfusion injury in a patient comprising; administering, a therapeutically effective amount of a polypeptide having at least 90% identity to the amino acid sequence of SEQ ID NO:2 over the entire length of SEQ ID NO:2 to the patient.

2. A method of (1) treating, healing or preventing wounds, osteoarthritis or rheumatoid arthritis; or (2) promoting cardiovascular tissue repair following reperfusion injury in a patient; comprising, administering a therapeutically effective amount of a polypeptide having at least 90% identity to the amino acid sequence of SEQ ID NO:4 over the entire length of SEQ ID NO:4 to the patient.

3. A method of (1) treating, healing or preventing wounds, osteoarthritis or rheumatoid arthritis; or (2) promoting cardiovascular tissue repair following reperfusion injury in a patient; comprising, administering a therapeutically effective amount of a polypeptide having the amino acid sequence of SEQ ID NO:2 to the patient.

4. A method of (1) treating, healing or preventing wounds, osteoarthritis or rheumatoid arthritis; or (2) promoting cardiovascular tissue repair following reperfusion injury in a patient; comprising, administering a therapeutically effective amount of a polypeptide having the amino acid sequence of SEQ ID NO:4.

5. A method of (1) treating, healing or preventing wounds, osteoarthritis or rheumatoid arthritis; or (2) promoting cardiovascular tissue repair following reperfusion injury in a patient; comprising, administering a therapeutically effective amount of a polypeptide having the amino acid sequence of SEQ ID NO:5.

6. A method of claim 1, 2, 3, 4 or 5 in which the wounds are selected from the group consisting of skin wounds, surgical wounds, burns, leg ulcers, diabetic ulcers, venous insufficiency ulcers, pressure ulcers, mucositis (both gastrointestinal and oral), renal fibrosis, lung fibrosis, COPD, and other lung diseases where damage to the epithelial cells and scar formation has occurred.

7. A pharmaceutical composition for (1) treating, healing or preventing wounds, osteoarthritis or rheumatoid arthritis; or (2) promoting cardiovascular tissue repair following reperfusion injury in a patient comprising a therapeutically effective amount of a polypeptide having at least 90% identity to the amino acid sequence of SEQ ID NO:2 over the entire length of SEQ ID NO:2.

8. A pharmaceutical composition for (1) treating, healing or preventing wounds, osteoarthritis or rheumatoid arthritis; or (2) promoting cardiovascular tissue repair following reperfusion injury in a patient comprising a therapeutically effective amount of a polypeptide having at least 90% identity to the amino acid sequence of SEQ ID NO:4 over the entire length of SEQ ID NO:4.

9. A pharmaceutical composition for treating or preventing (1) treating, healing or preventing wounds, osteoarthritis or rheumatoid arthritis; or (2) promoting cardiovascular tissue repair following reperfusion injury, in a patient comprising a therapeutically effective amount of a polypeptide having the amino acid sequence of SEQ ID NO:2.

10. A pharmaceutical composition for treating or preventing (1) treating, healing or preventing wounds, osteoarthritis or rheumatoid arthritis; or (2) promoting cardiovascular tissue repair following reperfusion injury in a patient comprising a therapeutically effective amount of a polypeptide having the amino acid sequence of SEQ ID NO:5.

11. A pharmaceutical composition of any one of claims 7-10 in which the wounds are selected from the group consisting of skin wounds, surgical wounds, burns, leg ulcers, diabetic ulcers, venous insufficiency ulcers, pressure ulcers, mucositis (both gastrointestinal and oral), renal fibrosis, lung fibrosis, COPD, and other lung diseases where damage to the epithelial cells and scar formation has occurred.
